(19) United States
(12) Patent Application Publication
(10) Pub. No.: US 2024/0173551 A1
(43) Pub. Date: May 30, 2024

(54) Closed-Loop Wireless Stimulation Systems with Wirelessly Powered Stimulators and Recorders

(71) Applicant: The Regents of the University of California, Oakland, CA (US)

(72) Inventor: Aydin Babakhani, Los Angeles, CA (US)

(73) Assignee: The Regents of the University of California, Oakland, CA (US)

(21) Appl. No.: 18/549,313

(22) PCT Filed: Mar. 10, 2022
(86) PCT No.: PCT/US2022/071066

§ 371 Date: Sep. 6, 2023

Related U.S. Application Data
(60) Provisional application No. 63/159,219, filed on Mar. 10, 2021.

(51) Int. Cl. A61N 1/36; A61N 1/02; A61N 1/372; A61N 1/378

(52) U.S. Cl. CPC A61N 1/36139; A61N 1/025; A61N 1/37235; A61N 1/37252; A61N 1/37288; A61N 1/3787

(57) ABSTRACT

Systems and methods for wirelessly powered biomedical treatment systems with closed-loop wireless implantable treatment devices for recording and stimulation are described. In an embodiment, a treatment system, includes: at least one implantable treatment device implantable in a location with respect to a body part, where the at least one implantable treatment device includes: an energy harvesting circuit configured to harvest ambient energy, a sensing circuit configured to sense bioelectrical signals, an stimulator circuit coupled to a set of electrodes to deliver energy, and a communication circuit configured to control the stimulation delivery circuit to deliver energy via at least one electrode from the set of electrodes in response to wireless control signals received from an external controller.

### CROSS REFERENCE TO RELATED APPLICATIONS

This application claims priority under 35 U.S.C. 119(e) to U.S. Provisional Patent Application Ser. No. 63/159,219, entitled “Closed-loop Wireless Stimulation Systems with Wirelessly Powered Stimulators and Recorders”, filed Mar. 10, 2021, the disclosure of which is incorporated herein by reference in its entirety.

### FIELD OF THE INVENTION

The present invention generally relates to biomedical treatment systems with closed-loop implantable treatment devices for sensing bioelectrical signals and stimulating an implant site.

### BACKGROUND

Biomedical implantable devices have solved various critical medical problems and improved the quality of human life. Their applications can include chronic pain relief, motor function recovery for spinal cord injuries, cardiac pacemaking, among various other applications. Conventional biomedical implantable devices can be bulky with the battery taking up most of the unit, and the necessary leads are prone to cause various complications.

### SUMMARY OF THE INVENTION

System and methods for biomedical treatment systems with closed-loop implantable treatment devices are described. In an embodiment, an implantable treatment device, includes: an energy harvesting circuit configured to harvest ambient energy, where the ambient energy is at least one energy selected from the group consisting of ambient electrical, magnetic energy, and electromagnetic energy; a sensing circuit that senses bioelectrical signals and generates bioelectrical signal data; a stimulator circuit coupled to a set of electrodes to deliver a stimulation via at least one electrode from the set of electrodes based on a set of control signals received from an external controller that determine a set of parameters of the stimulation; and a communication circuit configured to (1) transmit to the external controller the bioelectrical signal data and (2) control the stimulator circuit to deliver energy via at least one electrode from the set of electrodes in response to the control signals received from the external controller.

In a further embodiment, the bioelectrical signal is at least one signal selected from the group consisting of a neural signal, a neural LFP (local field potential), an electrocardiogram (ECG) signal, a compound action potential, an electromyogram (EMG) signal, an Electroencephalogram (EEG) signal, an Electromyogram (EMG) signal, an Electrooculogram (EOG) signal, an Electroretinogram (ERG) signal, and an Electrogastrogram (EGG) signal.

In a further embodiment, the sensing circuit digitizes the bioelectrical signals to generate bioelectrical signal data.

In a further embodiment, the bioelectrical signal data are used to optimize a plurality of parameters of the stimulation delivered by the stimulator circuit.

In a further embodiment, the plurality of parameters includes at least one parameter selected from the group consisting of the stimulation pulse duration, amplitude of the stimulation, frequency of the stimulation, and directionality and phase response of the stimulation (mono phasic vs biphasic).

In a further embodiment, the external controller processes the bioelectrical signal data to generate the set of control signals.

In a further embodiment, the implantable treatment device is a closed loop neural stimulation system.

In a further embodiment, the energy harvesting circuit is wirelessly powered.

In a further embodiment, the bioelectrical signals are neural signal that range from 1 uV to 100 Mv

In a further embodiment, the implantable treatment device further includes filter circuitry that filters the bioelectrical signals and digitizes the filtered bioelectrical signals.

In a further embodiment, the filter circuitry is designed to pass signals from 0.1 Hz to 10 KHz.

In a further embodiment, the filter circuitry passes more of a frequency content of the bioelectrical signal while removing out of band noise.

In a further embodiment, the bioelectrical signals are sensed within a preprogrammed time after a stimulation occurs.

In a further embodiment, the preprogrammed time sensing starts at a particular time after a stimulation occurs, where the particular time is a time selected from the group consisting of 1 usec, 10 usec, 100 usec, 1 msec, 10 msec, and 100 msec after the simulation occurs.

In a further embodiment, the sensing is done in an intermittent fashion with a duty cycle of less than 20%.

In a further embodiment, the stimulator circuit stimulates a body part in at least one body region selected from the group consisting of peripheral nerves, spinal cord, vagus nerve, central nervous system, sacral nerve, occipital nerve, hypoglossal nerve, kidney, bladder, brain, lung, heart, muscle, and fat.

In a further embodiment, the set of control signals provide several stimulation parameters for the stimulation that are optimized using processing performed in the cloud.

In a further embodiment, the control signals provide several stimulation parameters that are optimized for the stimulation using data from several different patients.

In a further embodiment, the control signals provide several stimulation parameters that are optimized for the stimulation with respect to a specific patient.

In a further embodiment, the energy harvesting circuit includes using energy stored within a rechargeable battery.

In a further embodiment, the energy harvesting circuit includes using energy from at least one capacitor.

In another embodiment, a treatment system, includes: an external controller; and a plurality of implantable treatment devices, each implantable treatment device configured for placement in, on, or adjacent an implant site, and comprising: a plurality of electrodes; a power harvesting circuit configured to receive energy from an external power source; a sensing circuit coupled to a sensing/recording set of the plurality of electrodes to sense bioelectrical signals and generate bioelectrical signal data; a stimulator circuit coupled to a stimulation set of the plurality of electrodes to deliver energy to the implant site; and a communication circuit coupled to the sensing circuit and the stimulator circuit and comprising a transmitter circuit configured to transmit to the external controller the sensed bioelectrical signal data, and a receiving circuit configured to receive stimulation control signals from the external controller and to control the stimulator circuit to deliver energy via the stimulation set of the plurality of electrodes according to stimulation control signals.

In a further embodiment, each implantable treatment device is configured for placement in, on, or adjacent an implant site corresponding to one of: a peripheral nerve, a spinal cord, a vagus nerve, a central nervous system, a sacral nerve, an occipital nerve, a hypoglossal nerve, a kidney, a bladder, a brain, a lung, a heart, a muscle, and fat.

In a further embodiment, the external controller includes: a bioelectrical data processing engine configured to process bioelectrical signal data received from at least one implantable treatment device; and a stimulation control data generator configured to generate stimulation control signals for at least one implantable treatment device based on the bioelectrical signal data.

In a further embodiment, the external controller further includes: a data transceiver configured to transmit the stimulation control signals to at least one implantable treatment device.

In a further embodiment, the external controller further includes: a power delivery circuit configured to deliver power to the plurality of implantable treatment devices.

In a further embodiment, the stimulation set of electrodes and the sensing/recording set of electrodes are spaced apart; and the sensing circuit is configured to: estimate a time window where a stimulation signal delivered by the stimulation set of electrodes arrives at the sensing/recoding set of electrodes, determine a time based on the estimated time window, and sense and record bioelectrical signals via the sensing/recording set of electrodes according to the determined time.

In a further embodiment, the determined time comprises one of 1 usec, 10 usec, 100 usec, 1 msec, 10 msec, and 100 msec.

In a further embodiment, the stimulation set of electrodes and the sensing/recording set of electrodes are spaced apart; and the sensing circuit is configured to: estimate a speed of the transfer of an activated neural signal from the location of the stimulation set of electrodes to the location of the sensing/recoding set of electrodes, determine a time based on the estimated speed, and sense and record bioelectrical signals via the sensing/recording set of electrodes according to the determined time.

In a further embodiment, the sensing circuit is configured to measure an evoked compound action potential (ECAP) based on bioelectrical signals sensed at the sensing/recording set of electrodes; the communication circuit is configured to transmit the measure of ECAP to the external controller; and the external controller is configured to adjust one or more stimulation parameters based on the ECAP, to maintain an appropriate level of neural activity.

In another embodiment, a method of therapy delivery by a treatment system, the method includes: sensing, through at least one implantable treatment device placed at an implant site, bioelectrical activity at the implant site; transmitting, through the at least one implantable treatment device, bioelectrical data corresponding to sensed bioelectrical activity to an external controller; receiving, through at least one implantable treatment device placed at an implant site, stimulation control data derived at least in part from the bioelectrical data by the external controller; and delivering, through the at least one implantable treatment device, a stimulation based on the stimulation control data.

In a further embodiment, the at least one implantable treatment device includes a stimulation set of electrodes spaced apart from a sensing/recording set of electrodes, and the method further comprising: estimating a time window where a stimulation signal delivered by the stimulation set of electrodes arrives at the sensing/recoding set of electrodes, determining a time based on the estimated time window, and sensing/recording bioelectrical signals via the sensing/recording set of electrodes according to the determined time.

In a further embodiment, the at least one implantable treatment device includes a stimulation set of electrodes spaced apart from a sensing/recording set of electrodes, and the method further comprising: estimating a speed of the transfer of an activated neural signal from the location of stimulation set of electrodes to the location of the sensing/recoding set of electrodes, determining a time based on the estimated speed, and sensing/recording bioelectrical signals via the sensing/recording set of electrodes according to the determined time.

In a further embodiment, the at least one implantable treatment device includes a stimulation set of electrodes spaced apart from a sensing/recording set of electrodes, and the method further comprising: measuring an evoked compound action potential (ECAP) based on bioelectrical signals sensed at the sensing/recording set of electrodes; transmitting the measure of ECAP to the external controller; and adjusting one or more stimulation parameters based on the ECAP, to maintain an appropriate level of neural activity.

In another embodiment includes an external treatment device configured for placement on a body part adjacent a treatment site, and including: a plurality of electrodes, a stimulator circuit coupled to the electrodes, and a receiving circuit configured to wirelessly receive stimulation control signals and to control the stimulator circuit to deliver energy via the plurality of electrodes according to stimulation control signals; at least one implantable treatment device configured for placement in, on, or adjacent a treatment site, and includes: a plurality of electrodes; an power harvesting circuit configured to receive energy from an external power source; a sensing circuit coupled to a sensing set of the plurality of electrodes to sense bioelectrical signals and generate bioelectrical signal data; and a communication circuit coupled to the sensing circuit and comprising a transmitter circuit configured to transmit the sensed bioelectrical signal data; and an external controller configured to receive the sensed bioelectrical signal data from the at least one implantable treatment device, and to transmit stimulation control signals to the external treatment device.

In a further embodiment, the external treatment device includes a sleeve configured to wrap around a patient's arm.

### BRIEF DESCRIPTION OF THE DRAWINGS

FIG. 1A illustrates a closed-loop treatment system with one or more implantable treatment devices for neural recording and stimulation in accordance with an embodiment of the invention.

FIG. 1B illustrates a closed-loop treatment system with one or more implantable treatment devices for neural recording and stimulation system in accordance with an embodiment of the invention.

FIG. 1C illustrates an implantable treatment device in accordance with an embodiment of the invention.

FIG. 1D illustrates a printed circuit board (PCB) of an implantable treatment device that includes a neurostimulation microchip and a recording microchip in accordance with an embodiment of the invention.

FIG. 1E illustrates an implantable treatment device with a neurostimulation microchip and a recording microchip in accordance with an embodiment of the invention.

FIG. 2A illustrates a treatment system that includes an external controller, several implantable treatment devices, and one or more cloud servers in accordance with an embodiment of the invention.

FIG. 2B illustrates a treatment system that includes an external controller and several implantable treatment devices in accordance with an embodiment of the invention.

FIG. 3 illustrates a system architecture of an implantable treatment device for bioelectrical recording and stimulation in accordance with an embodiment of the invention.

FIG. 4 illustrates a system architecture of an external controller for processing bioelectrical data and generating stimulation control signals to be transmitted to one or more implanted treatment devices in accordance with an embodiment of the invention.

FIG. 5A illustrates a process for recording and transmitting bioelectrical data in an implanted treatment device in accordance with an embodiment of the invention.

FIG. 5B illustrates a process for receiving and performing stimulation in an implantable treatment device in accordance with an embodiment of the invention.

FIG. 6 illustrates a process for processing bioelectrical data within an external controller and transmitting the control signals to an implantable treatment device in accordance with an embodiment of the invention.

FIG. 7 illustrates a timing diagram for performing stimulation and then sensing and recording bioelectrical data after a particular pre-programmed time period after the stimulation in accordance with an embodiment of the invention.

FIG. 8A illustrates a circuitry overview, with the circuit architecture of an implantable treatment device in accordance with an embodiment of the invention.

FIG. 8B illustrates a schematic of the Tx coil in accordance with an embodiment of the invention.

FIG. 9 illustrates a closed-loop treatment system for neural recording and stimulation for neural prostheses in accordance with an embodiment of the invention.

### DETAILED DESCRIPTION OF THE DRAWINGS

Turning now to the drawings, closed-loop treatment systems for recording and stimulation in accordance with various embodiments of the invention are illustrated. Further details of the closed-loop recording and stimulation systems are discussed in U.S. Provisional Patent Application 63/127,702 entitled “Wireless Recording System-on-chip for Distributed Neural Interface Systems with Inductive Power Delivery and UWB Data Transmission” by Rahmani et al., and U.S. PCT Application PCT/US2021/020343 entitled “Integrated Energy Harvesting Transceivers and Transmitters With Dual-Antenna Architecture for Miniaturized Implants and Electrochemical Sensors” by The Regents of the University of California et al., and U.S. PCT Application Number PCT/US2020/048001 entitled “Wirelessly Powered Stimulator”, by The Regents of the University of California et al., the entirety of which are herein incorporated by reference.

Emerging applications of Brain Machine Interface (BMI) systems show an ever-increasing demand for neural activity acquisition with higher data-rates and better spatiotemporal resolutions. Accordingly, many embodiments provide for a treatment system that includes an external controller wirelessly communicating with at least one implantable treatment device implantable in a location with respect to a body part, including on, within and/or proximate a tissue. In many embodiments the implantable treatment device can be a brain implant that is implanted to sense and record neural activity and stimulate electrodes on the brain and/or electrodes on different body parts. In certain embodiments, the treatment system including the one or more implantable treatment device can be for any of a variety of biomedical implant applications, including pace makers, brain implants, spinal cord implants, among various other applications.

In many embodiments, the treatment system can include an external controller that wirelessly communicate and/or provides wireless power to one or more implantable treatment devices. In certain embodiments, the external controller can include a rechargeable battery that provides power and/or communicates thru wiring to one or more implantable treatment devices. In several embodiments, the external controller can be a wearable device worn on a body part of a patient. In certain embodiments, the external controller can be a software application executing on a computer, smartphone application, among other types of devices, and/or one or more cloud servers.

In many embodiments, the treatment system can include an external controller communicating with an array of implantable treatment devices, where each implantable treatment device includes a semiconductor substrate that includes a communication circuit, an energy harvesting circuit, and a stimulation delivery circuit. In many embodiments, an array of wirelessly powered implantable treatment devices can be placed at different spatial locations on a body part (e.g., brain, heart, among various others) and each implantable treatment device can sense and/or stimulate a specific location, and wirelessly communicate with a centralized external controller in order to provide the controller with the sensed activity and to receive control signals from the external controller regarding various stimulation parameters. Accordingly, unlike many prior art brain implants and heart implants that placed electrodes within a confined spatial location that required wiring between a controller and the recording/stimulation sites, many embodiments are able to place the recording and stimulation sites at any location on a body part without wiring.

In many embodiments, an implantable treatment device can include several circuits including an energy harvesting circuit, a stimulation delivery circuit and a communication circuit. In many embodiments, the energy harvesting circuit can be configured to harvest ambient energy, where the ambient energy can be ambient electrical, magnetic energy, and/or electromagnetic energy. The implantable treatment device can include a stimulation delivery circuit coupled to a set of electrodes to deliver energy to an implant site, and a communication circuit configured to control the stimulation delivery circuit to deliver energy via at least one electrode from a set of electrodes. In many embodiments, the stimulation delivery circuit can deliver energy via the electrodes in response to wireless control signals received from an external controller, where the control signals provide a variety of different parameters of the stimulation. In particular, in many embodiments, the implantable treatment device can wirelessly transmit sensed and recorded bioelectrical signals (e.g., neural signals from a brain, bioelectrical signals from a heart, among other types of bioelectrical signals obtained from different body parts) activity to an external controller and the external controller can process the recorded bioelectrical activity and transmit an optimized set of stimulation control signals to the implantable treatment device to stimulate an implant site based on the sensed bioelectrical signal activity. The bioelectrical signals may also be referred to and discussed herein as neural signals, neural LFP (local field potential), an electrocardiogram (ECG) signal, compound action potential, an electromyogram (EMG) signal, an Electroencephalogram (EEG) signal, an Electromyogram (EMG) signal, an Electrooculogram (EOG) signal, an Electroretinogram (ERG) signal, and/or an Electrogastrogram (EGG) signal, among various other types of bioelectrical signals.

In many embodiments, the implantable treatment device can be wirelessly powered to sense and record bioelectrical (e.g., neural, cardiac, among various others) activities. The sensing can be done before, during, and/or after stimulation. The implantable treatment device can digitize a bioelectrical signal prior to processing the bioelectrical signal. The digitized bioelectrical (e.g., neural, cardiac, etc.) signal can be processed in the implantable treatment device. In certain embodiments, the implantable treatment device can include a wirelessly powered transmitter, where a digitized signal is transmitted wirelessly to an external controller using the wirelessly powered transmitter. A sensed bioelectrical signal and bioelectrical activity can be used to optimize a plurality of parameters of the stimulation delivery circuit of the implantable treatment device. In many prior art applications, the placement of a stimulation contact can be wrong, or the quality of the contact is low. In some prior art devices, more than one electrode has been used to increase the redundancy and improve the results. However, redundancy can fail and thus is not sufficient in many applications. To address this issue, many embodiments are able to demonstrate that a stimulation is happening, and that neurons get activated. Accordingly, many embodiments use a sensing/recording node to measure the quality of a stimulation. Many embodiments are able to achieve this by placing a sensing/recording node at a set distance from a stimulation node. In embodiments where sensing/recording and stimulation are enabled through electrodes, a sensing/recording node may be defined by a first set electrodes and a stimulation node may be defined by a second set of electrodes. By estimating the speed of the transfer of the neural signal in the body, many embodiments are able to estimate a time window where the stimulation signal arrives at the sensing/recoding node. This can help to verify that neurons are activated and also verify that the activation of the neurons are due to the stimulation waveform. Accordingly, an implantable treatment device can sense and measure the evoked compound action potentials (ECAPs) and then an external controller can automatically adjust one or more stimulation parameters, including the stimulation amplitude (e.g., current or voltage) to maintain an appropriate level of neural activity. In many embodiments, the sensing can help to optimize a dose of the stimulation and also identify the quality of the contact with the tissue and the neurons.

In certain embodiments, the SNR measured at a sensing/recoding node may not be sufficient. To address this issue, many embodiments may repeat the stimulation and re-measure the neural activity at a sensing/recording node and use averaging of the sensed/recorded signals to improve SNR.

In many embodiments, the treatment system can optimize a set of power delivery and power consumption parameters based on characteristics of an implantable treatment device at a particular implant location. For example, the treatment system can minimize and/or optimize the power consumption settings of an implantable treatment device based on detected characteristics of the operation of the implantable treatment device at a particular implant site. For example, for an implantable treatment device implanted deep within a tissue, the implantable treatment device may consume more power to send wireless data to the external controller than an implantable treatment device located near the surface of a tissue. Accordingly, the treatment system can detect, based on measured characteristics of the bioelectrical sensing data and stimulation data, an optimal set of power consumption settings and stimulation parameters for a particular implantable treatment device at a particular implant location using real-time operation data of the implantable treatment device.

In many embodiments, the bioelectrical activity from different implantable treatment devices in a particular patient can be used to determine one or more parameters for a particular treatment device in the patient. The parameters can include parameters for the stimulation delivery circuit of an implantable treatment device, including a stimulation pulse duration, amplitude of a stimulation, frequency of a stimulation, and directionality and phase response of a stimulation (e.g., mono phasic vs biphasic), among various other stimulation parameters. The external controller can process the bioelectrical signals recorded by the implantable treatment device to provide the control signals. The control signals can determine the stimulation parameters of the implantable treatment device, including the particular set of one or more implantable treatment devices and/or electrodes on the body that should be stimulated. In several embodiments, the implantable treatment device is a closed loop neural stimulation system. In many embodiments, the stimulation delivery circuit, energy sensing circuit, and/or an array of implantable treatment devices that sense and record bioelectrical activity and/or stimulate a tissue can be wirelessly powered. In many embodiments, the recorded neural signal can range from 1 uV to 100 mV. The recorded signal can be filtered before being digitized.

In many embodiments, the filter can be designed to pass signals from 0.1 Hz to 10 KHz. Accordingly, the filter may pass more of a frequency content of the neural signal while removing out of band noise.

In order to improve the quality of the recorded bioelectrical signals, many embodiments may time a stimulation according to the sensed neural activity. In particular, the neural signals may be sensed within a preprogrammed time after a stimulation occurs. This time can be determined by estimating the speed of the transfer of the activated neural signal from the location of the stimulation to the location of the sensing/recoding node.

In several embodiments, the preprogrammed time sensing starts at a particular time after a stimulation occurs, where the particular time can be at 1 usec, 10 usec, 100 usec, 1 msec, 10 msec, or 100 msec after the simulation occurs. In certain embodiments, the sensing can be done in an intermittent fashion with a duty cycle of less than a certain percentage. In certain embodiments, the duty cycle can be less than 20%. For example, sensing can be performed for 200 msec every 1 sec.

Many embodiments of the treatment system utilize energy harvesting and thus operate using a duty-cycling approach. The implantable treatment devices may turn on only for a certain percentage of the time (e.g., 1-10% of the time). For example, a recording or stimulation node may harvest 1 uW of average power and use it for only 1% of the time. This duty-cycling approach may allow the recording or stimulation nodes to boost the effect power by the inverse of the duty cycle. A recording and/or stimulation node can consume a certain amount of power for a certain period of time. For example, a recording and/or stimulation node can consume 100 uW DC power only for 1% of the time, while harvesting only 1 uW on average. Further details of the duty-cycling approach for both sensing and stimulation nodes are discussed in details in U.S. Provisional Patent Application 63/127,702 entitled “Wireless Recording System-on-chip for Distributed Neural Interface Systems with Inductive Power Delivery and UWB Data Transmission” by Rahmani et al., and U.S. PCT Application PCT/US2021/020343 entitled “Integrated Energy Harvesting Transceivers and Transmitters With Dual-Antenna Architecture for Miniaturized Implants and Electrochemical Sensors” by The Regents of the University of California et al., and U.S. PCT Application Number PCT/US2020/048001 entitled “Wirelessly Powered Stimulator”, by The Regents of the University of California et al., the entireties of which are herein incorporated by reference.

In many embodiments, the implantable treatment device can be implanted in various different locations in a body without the need for intrusive wiring and bulky components. In particular, an array of implantable treatment devices can be positioned at any of a variety of locations and can each communicate with a centralized external controller to transmit recorded bioelectrical signals and to receive control signals regarding the stimulation parameters for stimulating a particular body part. The stimulation delivery circuit of an implantable treatment device can stimulate a body part that can include peripheral nerves, spinal cord, vagus nerve, central nervous system, sacral nerve, occipital nerve, hypoglossal nerve, kidney, bladder, brain, lung, heart muscle, and fat. In many embodiments, the stimulation and sensing in a particular body part can be used to optimize a plurality of stimulation parameters. In many embodiments, a first implanted treatment device located at a particular body part can sense and record signals and transmit the signals to the external controller. The external controller can process this and/or other signals from other implantable treatment devices, and transmit control signals to a second implantable treatment device located at a different location to stimulate a body part. Accordingly, different implantable treatment devices can sense and send signals to a centralized external controller that can process the various bioelectrical signals and determine optimization parameters for one or more different implantable treatment devices that can stimulate different locations of a body. In many embodiments, the external controller can use data from a particular patient to determine the optimization for the control signals. In several embodiments, the external controller can use data from different patients to determine the control signals. In particular, recorded bioelectrical signals (e.g., neural signals) can be processed using different patient data in order to determine the optimization stimulation parameters of a particular patients. In many embodiments, the external processing and optimizations can be done on the cloud, where information can be combined from multiple patients and one or more servers can perform the optimization. In certain embodiments, the optimization can be patient specific and the processes can be tailored to specific patient data.

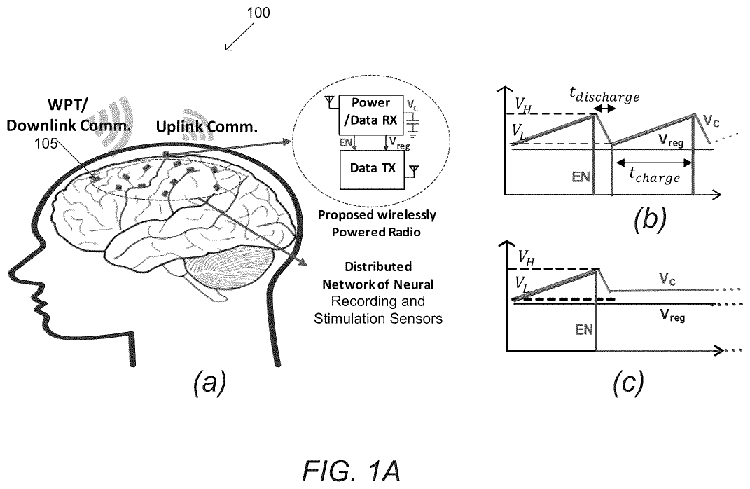

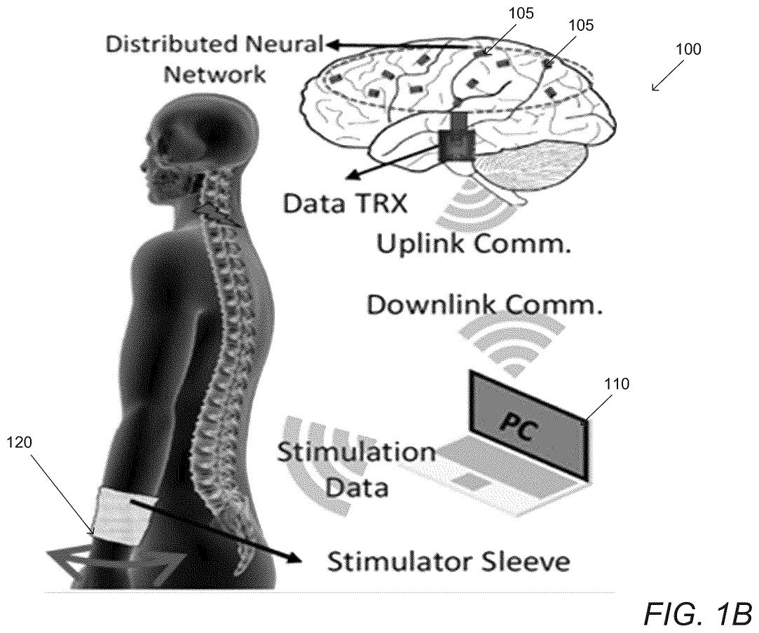

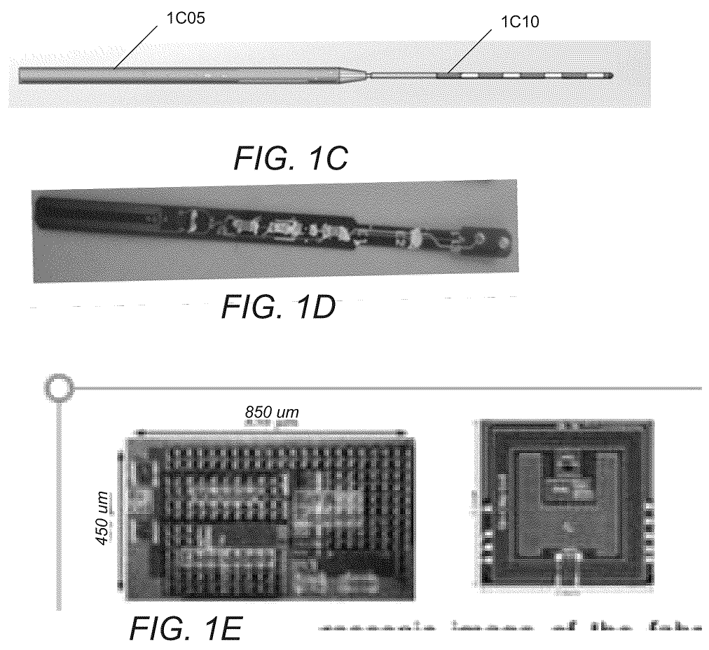

A closed-loop treatment system 100 with several implantable treatment devices 105 for neural recording and stimulation in accordance with an embodiment of the invention is illustrated in FIG. 1A and FIG. 1B. Neural signals (e.g., brain signals) can be sensed and recorded and nerves can be stimulated by a treatment system 100 that includes several implantable treatment devices 105 distributed at different locations on a body part and the data may be transmitted to an external processor 110 wirelessly. As illustrated, the treatment system can be used for neural sensing an stimulation and may comprise an array of implantable treatment devices 105 positioned at different locations on the brain, wherein each implantable treatment device 105 can be wirelessly powered, wirelessly sense and transmit bioelectrical (e.g., neural) signals to an external controller 110, and wirelessly receive control signals from the external controller 110 to determine a set of optimal stimulation parameters for the implantable treatment device and to stimulate one or more electrodes implanted on the brain. An implantable treatment device can include a semiconductor substrate that includes a communication circuit, an energy harvesting circuit, a stimulation delivery circuit, and/or a sensing circuit. After the neural signals are decoded, different body parts can be stimulated by electrodes, as illustrated in FIG. 1B, arm muscles can be stimulated by an electronic sleeve 120 wrapped around a patient's arm in order to help the patient regain control over their hand movement. Providing a real-time hand control thru stimulation is made possible in that many embodiments are able to acquire neural activity with a high throughput at a fine scale.

In a treatment system with a distributed network of implantable sensor devices for sensing and stimulation as illustrated in FIGS. 1A and 1B, miniaturizing the individual implantable treatment devices 105 can be an effective solution that renders more recording and stimulation sites and improves the resolution. Accordingly, many embodiments provide for a treatment system with a distributed network of implantable treatment devices based on a fully integrated system-on-chip (SoC). Further discussion of circuit designs that may be utilized for implantable treatment devices for neural recording and stimulation are described in U.S. Provisional Patent Application 63/127,702 entitled “Wireless Recording System-on-chip for Distributed Neural Interface Systems with Inductive Power Delivery and UWB Data Transmission” by Rahmani et al., and U.S. PCT Application PCT/US2021/020343 entitled “Integrated Energy Harvesting Transceivers and Transmitters With Dual-Antenna Architecture for Miniaturized Implants and Electrochemical Sensors” by The Regents of the University of California et al., and U.S. PCT Application Number PCT/US2020/048001 entitled “Wirelessly Powered Stimulator”, by The Regents of the University of California et al., the entireties of which are herein incorporated by reference.

In many embodiments, the treatment system is able to improve the spatiotemporal resolution of the recorded bioelectrical signals to provide more insight into the complex mechanism of human functions. To enable recording and/or sensing at a fine scale, many embodiments of the invention utilize implantable System-on-Chips (SoCs) to realize a distributed treatment system for neural recording. The system-level requirements of such SoCs are long-term wireless operation, mm-sized form-factor, and integration capability on a commercial CMOS process to make them scalable and cost-efficient. Miniaturizing the size of an implantable treatment device can be a key step for fulfilling the needs of next-generation biomedical treatment systems with implantable treatment devices since it results in a higher sensor density and also enables signal recording at an ultra-small structural scale. FIG. 1A illustrates a conceptual multi-site and distributed treatment system 100 enabled by an array of mm-sized implantable treatment devices 105 for recording and/or stimulation in accordance with an embodiment of the invention. Each implantable treatment device 105 can include a data transceiver (TRX) that is placed on top of a microelectrode array. Due to the compact size, individual treatment devices 105 can be tightly placed on the brain or other tissues and thus improve the recording resolution. There can be many challenges toward realizing a practical neural interface system. In many embodiments, each TRX can act as a communication hub between the electrodes within an implantable treatment device 105 and an external reader 110.

Many embodiments of the treatment system described here present the design, implementation, and verification of a fully integrated and RF-powered wireless implantable treatment device. The implantable treatment device can be implemented on a single CMOS silicon chip and include the various components for power delivery, energy storage, bioelectrical sensing, stimulation, and data communication, including an on-chip coil and a dipole antenna, can be implemented on the same chip. In certain embodiments, some components such as antennas can be located off chip.

Although specific implementations for closed-loop wireless treatment system with wirelessly powered implantable treatment devices for recording and stimulation are discussed above with respect to FIG. 1A and FIG. 1B, any of a variety of implementations utilizing the above discussed techniques can be utilized as appropriate to the requirements of specific applications in accordance with embodiments of the invention.

FIG. 1C illustrates an implantable treatment device in accordance with an embodiment of the invention. As illustrated, the implantable treatment device can be a neuro-stimulator. The implantable treatment device can include a housing 1C05 connected to an electrode 1C10. Within the housing 1C05 can be a printed circuit board that includes a variety of microchips and circuitry. FIG. 1D illustrates a PCB of an implantable treatment device that includes a neurostimulation microchip, a recording microchip, and various other circuitry. FIG. 1E illustrates a neurostimulation microchip and a recording microchip in accordance with an embodiment of the invention. Although FIG. 1C-1E illustrate a particular implantable treatment device for neurostimulation and recording, any of a variety of implantable treatment device architectures can be utilized as appropriate to the requirements of specific applications, including applications to different body parts and types of bioelectrical activity in accordance with embodiments of the invention.

### Treatment System Architecture

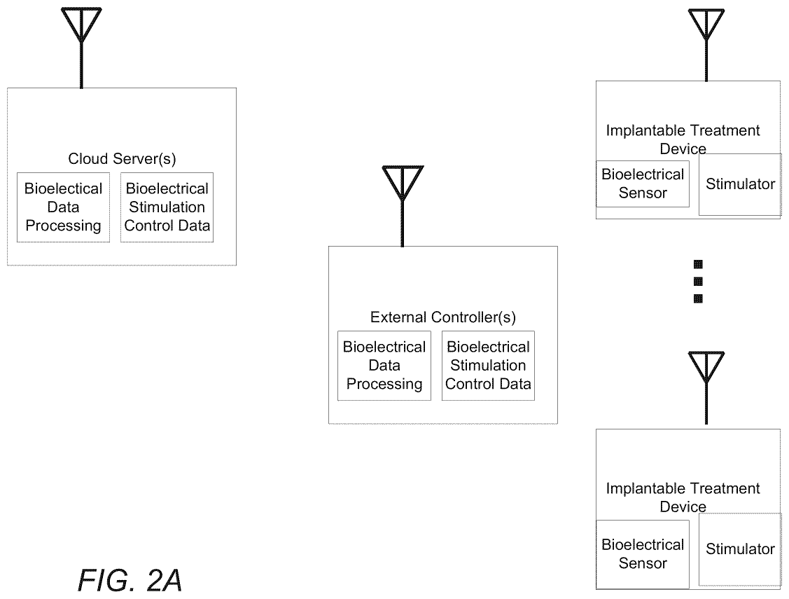

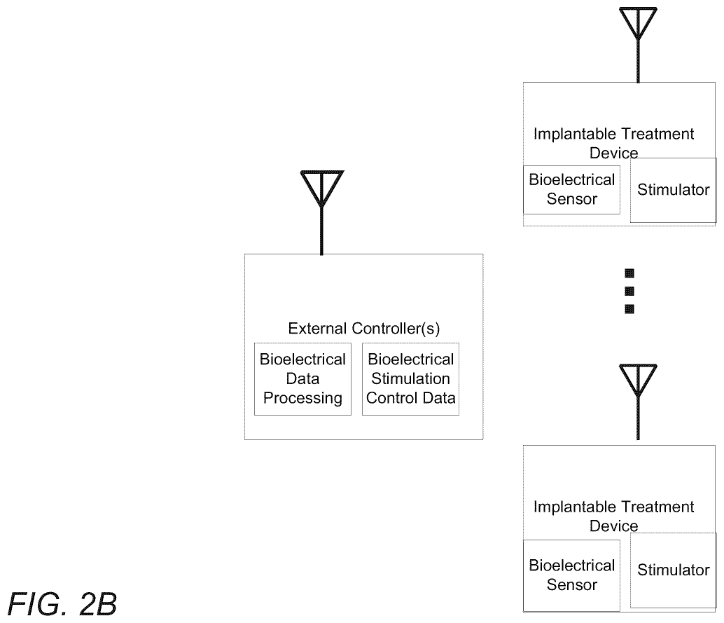

A system architecture diagram of a treatment system in accordance with several embodiments of the invention is depicted in FIG. 2A. The treatment system can include one or more cloud servers, one or more external controllers, and one or more implantable treatment devices. Each of the implantable treatment devices can be implanted at different body part locations within a patient. An implantable treatment device can include a bioelectrical sensor and/or recorder than can sense and/or record bioelectrical signal activity and transmit the bioelectrical data to the external controller. An implantable treatment device can include a stimulator circuit than can deliver an energy to stimulate an implant location. In many embodiments, the implantable treatment device can wirelessly communicate with the external controller. In many embodiments, the implantable treatment device can be wirelessly powered. The wireless power can be received from the external controller. In certain embodiments, the implantable treatment device can be wired to receive power and/or communicate with the external controller. In certain embodiments, the implantable treatment devices and/or external controller can include rechargeable batteries that can be charged wirelessly and/or through wiring.

The external controller can receive bioelectrical signal data from one or more implantable treatment devices and can process the bioelectrical signal data. In many embodiment, the external controller can process the bioelectrical signal data to determine an optimal set of stimulation parameters for performing a stimulation by a particular implantable treatment device. The external controller can transmit the bioelectrical control data to one or more implantable treatment devices and the implantable treatment device can stimulate an implant location accordingly. The external controller can also communicate with one or more cloud servers, and the cloud servers can also process the bioelectrical data and/or generate optimized bioelectrical stimulation control data. Accordingly, many embodiments of the treatment system are able to minimize the power consumption of the implantable treatment devices by performing the more extensive power consuming processing of the bioelectrical signal data on the external controller and/or the cloud servers. Accordingly, the implantable treatment devices can minimize the power necessary to enable their operations.

FIG. 2B illustrates a treatment system that includes an external controller and several implantable treatment devices in accordance with an embodiment of the invention. Although FIG. 2A and FIG. 2B illustrate specific system architectures of treatment systems for recording bioelectrical data and stimulation of an implant site, any of a variety of architectures can be utilized as appropriate to the requirements of specific applications in accordance with embodiments of the invention.

### Implantable Treatment Device System Architecture

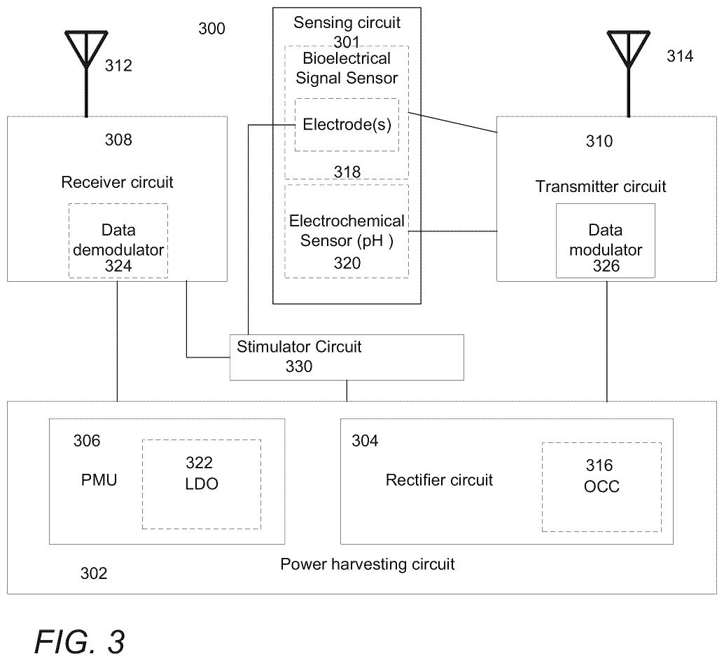

A block diagram of an implantable treatment device in accordance with several embodiments of the invention is depicted in FIG. 3. The implantable treatment device 300 can include a power harvesting system 302, which includes a rectenna (rectifier circuit) 304 and a power management unit (PMU) 306, a receiver circuit (RX) 308, and a transmitter circuit (TX) 310. A receive antenna 312 is connected to the receiver circuit 308 and a transmit antenna 314 is connected to the transmitter circuit 310.

The rectenna 304, which may include an on-chip coil (OCC) 316, several full-wave rectifiers and a matching capacitor, can receive energy through an inductive link and convert RF energy into a DC voltage. The OCC can be shared between the power harvesting system 302 (for power) and the receiver 308 (for receiving data). The converted power by the rectenna can be used to power other components of the implantable treatment device 300 such as the data transmitter.

The receiver circuit 308 may include a data demodulator 324 to receiver data at the implantable treatment device. Some embodiments may not receive data and therefore may not have a data decoder in the receive circuit. Some embodiments may extract a clock signal from the received signal. In some embodiments, the receive antenna 312 is a loop antenna with a capacitor to utilize resonance inductive coupling. In other embodiments, the receive antenna 312 is a dipole antenna and other configurations maybe contemplated.

In several embodiments, the transmitter 310 includes a reconfigurable data modulator circuit 326 to send data out from the implantable treatment device. In different embodiments of the invention, the transmit antenna 314 can be a monopole, dipole, or loop antenna as appropriate to a particular application, although isolation from the receive antenna 312 is desirable.

The main power-consuming block of the implantable treatment device can be often the transmitter (TX) 310. Due to the challenges of power transfer to mm-sized implants, harvested power is often less than the instantaneous power consumption of the TX block 310. Therefore, power management unit (PMU) 306 can duty-cycle the operation of the data TX 310 to maintain a minimum voltage across the storage capacitor (C_{S}) and establish charging and discharging modes for C_{S}. In charging mode, the converted power by the rectenna increases the voltage level across C_{S} (V_{C}) until the PMU 306 activates the TX block 310. PMU 306 and data receiver (RX) 308 blocks can be active during the entire operation and constituent sub-circuits are designed in subthreshold region to maximize sensitivity and reduce the charging time (t_{charge}) of C_{S}. On the other hand, discharging time (t_{discharge}) of C_{S} is proportional to the capacitance value; hence using a large capacitance enables the PMU to follow rapid transitions of V_{C}. In several embodiments of the invention, in order to achieve a high capacitance density, MIM capacitors (2 fF/μm2) are stacked over MOSCAP devices (5.5 fF/μm2) to realize a 5 nF capacitor, although other designs may be utilized to achieve a target capacitance. The transition from charging mode to discharging mode represents a significant load variation for the low dropout voltage regulator (LDO) 322 in the PMU 306. To ensure the regulator remains functional, the bandwidth of the error amplifier can be increased at the onset of active mode. The PMU 306 can adaptively change the bias condition of the LDO and enables it to maintain a constant voltage at its output.

In several embodiments of the treatment system, an implantable treatment device for a bioelectrical recording and stimulation application (e.g., neural recording and stimulation) based on sensed bioelectrical activity can receive information from a sensing circuit 301 that includes a bioelectrical signal sensor 318 that includes a set of sensing/recording electrodes and stimulate a body part (e.g., nerve, tissue, among various other body parts) using stimulator circuit 330 (e.g., an energy delivery circuit) connected to a set of stimulating electrodes. A bioelectrical signal sensor 318 can include any of a variety of sensors, configured to capture electrical signals from which one or more of the following can be derive: neural LFP (local field potential), an electrocardiogram (ECG) signal, compound action potential, an electromyogram (EMG) signal, an Electroencephalogram (EEG) signal, an Electromyogram (EMG) signal, an Electrooculogram (EOG) signal, an Electroretinogram (ERG) signal, and/or an Electrogastrogram (EGG) signal. Such sensors sense electrical signals, potential, or other characteristics in a variety of ways such as the difference between two electrodes, electrical resistance, or the magnetic field induced by electrical currents. Such sensing applications may complement neural stimulation for a variety of therapies, such as pain control.

In some other embodiments of the invention, a transmitter for electrochemical sensing can include an electrochemical sensor 320, such as a pH sensor. Such sensors may be used in industrial or environmental applications.

In many embodiments, a 250 MHz signal is utilized to power the implantable treatment device by received signal as it provides high penetration, and higher harmonics of this frequency can cause interference. Additionally, the received power signal may utilize amplitude modulation. In several embodiments, a 4.15 GHz center frequency is utilized for the transmit signal to provide high bandwidth and avoid harmonics of the receiver frequency. Frequencies should be utilized that are far from each other to be more isolated and decouple interference between the receive power link and the transmit uplink. A treatment system in accordance with embodiments of the invention may adaptively set transmit mode and/or data rates and utilize variable power, rather than target a specific power budget and data rate.

Although specific implantable treatment devices are described above with respect to FIG. 3, one skilled in the art would recognize that certain components of the implantable treatment device described above may be different, may have different characteristics, or be different in number in accordance with embodiments of the invention as appropriate to the requirements of a particular application in accordance with embodiments of the invention.

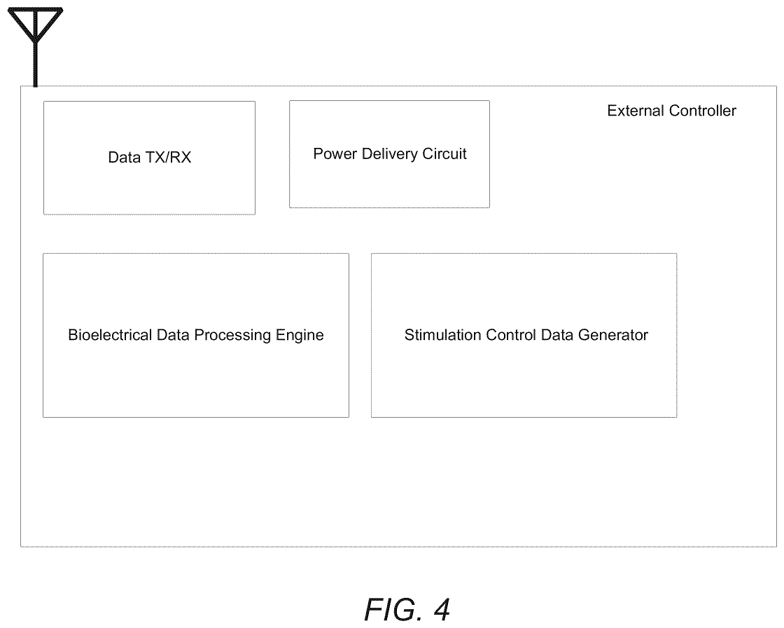

FIG. 4 illustrates a circuit architecture of an external controller in accordance with an embodiment. The external controller can include a data receiver and/or transmitter (TX/RX), a power delivery circuit, a bioelectrical data processing engine, and a stimulation control data generator. The external controller can receive bioelectrical e.g., neural data from one or more implantable treatment devices. The received neural data can be processed. One or more stimulation parameters can be generated based on the processed neural signal data. The data TX/RX can transmit the stimulation control signals to one or more implantable treatment devices in order to perform a stimulation. The power delivery circuit can provide power to one or more implantable treatment devices. In certain embodiments, the power can be delivered wirelessly to the implantable treatment devices. In several embodiments, the power can be delivered through wires to the implantable treatment devices.

Although FIG. 4 illustrates a particular circuit architecture of an external controller, any of a variety of system architectures can be utilized as appropriate to the requirements of a specific application in accordance with embodiments of the invention.

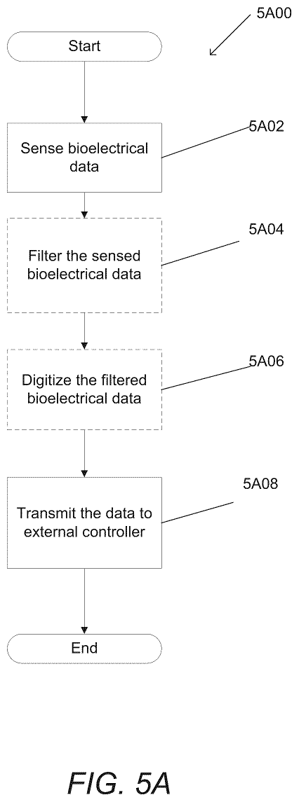

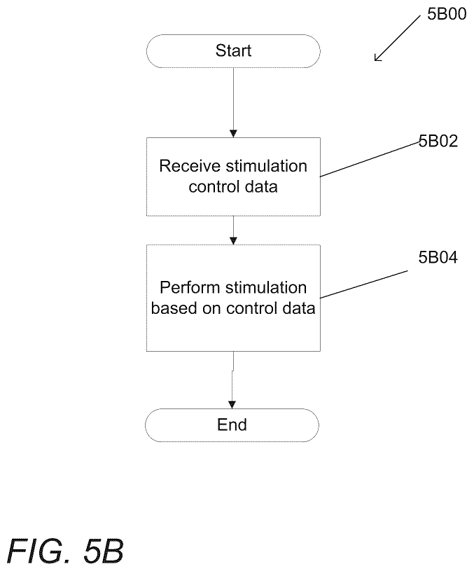

A process for sensing/recording neural data by an implantable treatment device and transmitting the data to an external controller in accordance with an embodiment of the invention is illustrated in FIG. 5a. The process can sense 5a02 and record bioelectrical signals. In certain embodiments, the process can filter 5a04 the recorded and/or sensed bioelectrical signal. The process can digitize 5a06 the bioelectrical signal into corresponding bioelectrical data. The process transmits 5a08 the bioelectrical data to an external controller. Although specific processes for recording and transmitting bioelectrical data are described above, any of a variety of processes may be utilized to record and transmit bioelectrical data as appropriate to the requirements of a specific application in accordance with embodiments of the invention.

A process for receiving stimulation control data at an implantable treatment device from an external controller and performing a stimulation in accordance with an embodiment of the invention is illustrated in FIG. 5b. The process 5b00 receives 5b02 stimulation control data from an external controller. In many embodiments, the control data can include one or more different stimulation parameters, including stimulation delivery circuit stimulation pulse duration, amplitude of the stimulation, frequency of the stimulation, and directionality and phase response of the stimulation (mono phasic vs biphasic). The process can perform a stimulation 5b04 based on the stimulation control data. The process ends. Although specific processes for performing a stimulation are described above, any of a variety of processes may be utilized perform stimulations as appropriate to the requirements of a specific application in accordance with embodiments of the invention.

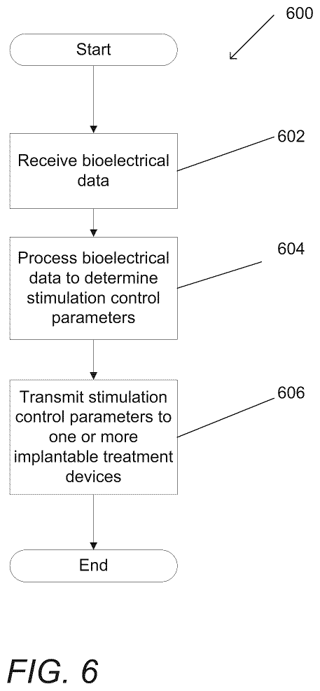

A process for generating stimulation control data on an external controller and transmitting the data to one or more implantable treatment devices in accordance with an embodiment of the invention is illustrated in FIG. 6. The process 600 can receive 602 sensed bioelectrical data from one or more implantable treatment devices. The process can process 604 the bioelectrical signal data to determine one or more stimulation control parameters. The process can transmit 606 the stimulation control parameters to one or more implantable treatment devices. The process ends.

In many embodiments, the treatment system can sense bioelectrical signal data after a particular time period after a stimulation has occurred. The time period can be pre-programmed. In several embodiments, the time period can be dynamic based on the measured signal recording and stimulation data.

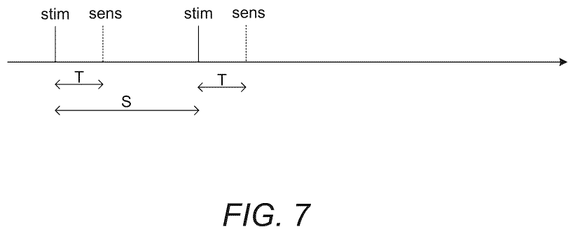

A timing diagram illustrating sensing bioelectrical data after a time period after stimulation is illustrate in FIG. 7. As illustrated, an external controller can periodically transmit a stimulation control signal to direct an implantable treatment device to deliver a stimulation. In the timeline of FIG. 7, the external controller controls the microchip to deliver stimulation (stim) every S seconds. In certain embodiments, S may be in the range of 1 usec to 100 msec. The stimulation may comprise a single electrical pulse or a train of electrical pulses. The external controller can also send a sense control signal to direct an implantable treatment device to sense (sens) the bioelectrical signals within a preprogrammed time (T) after the stimulation occurs. In certain embodiments, T may be in the range of 1 usec to 100 msec. In many embodiments, this time (T) can be determined by estimating the speed of the transfer of the activated neural signal from the location of the stimulation to the location of the sensing node.

In several embodiments, the preprogrammed time sensing starts at a particular time after a stimulation occurs, where the particular time can be at 1 usec, 10 usec, 100 usec, 1 msec, 10 msec, or 100 msec, among various other set times after the simulation occurs. In certain embodiments, the sensing can be done in an intermittent fashion with a duty cycle of less than a certain percentage. In certain embodiments, the duty cycle can be less than 20%. For example, sensing can be performed for 200 msec every 1 sec. Although a specific timing diagram for performing a sensing after a stimulation occurs is illustrated in FIG. 7, any of a variety of timing specifications can be utilized as appropriate to the requirements of specific applications in accordance with embodiments of the invention.

### System Architecture of Implantable Pulse Generator

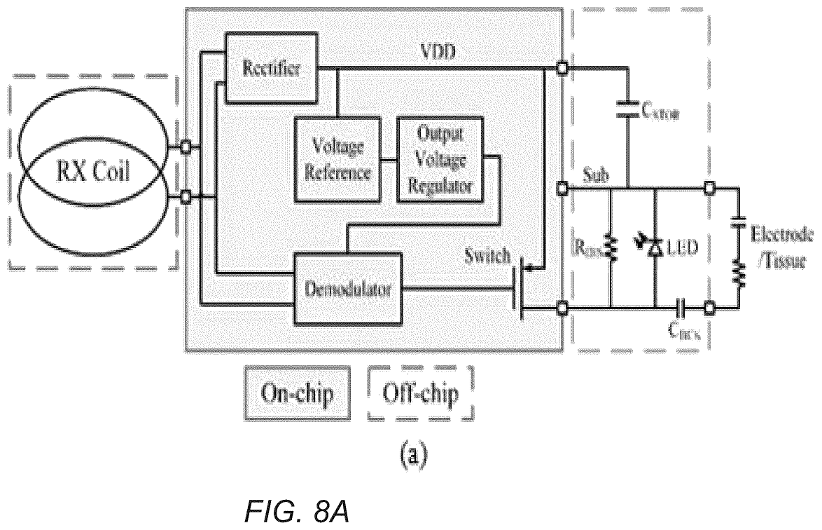

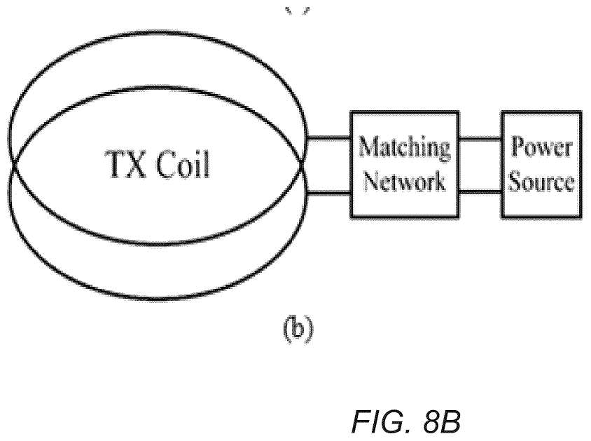

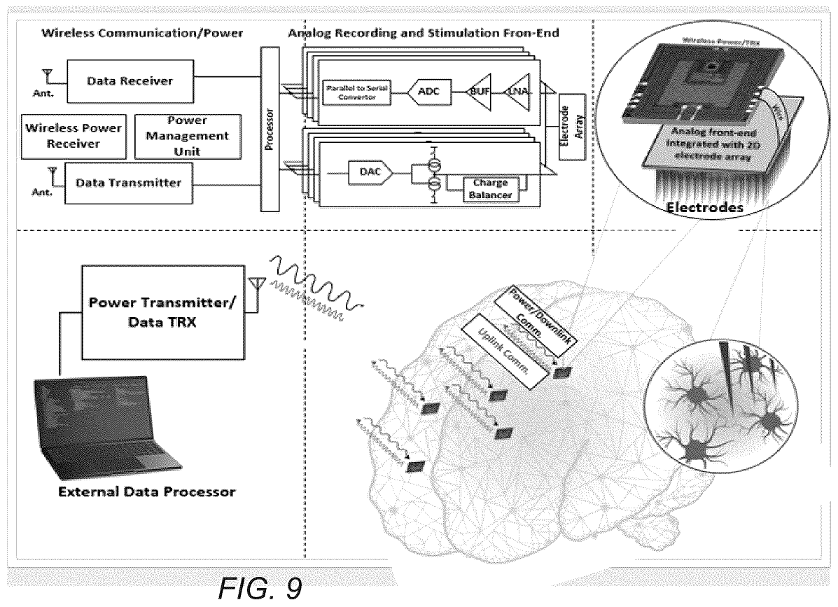

A system architecture of an implantable treatment device in accordance with an embodiment of the invention is shown in FIG. 8a. In many embodiments, the magnetic field coupled to the Rx coil can be rectified to generate VDD and charges an energy storage capacitor, CSTOR. In several embodiments, notches (e.g., RF power is reduced to a percentage of the RF power during harvest) can be intentionally applied in the Tx signal which precisely controls the timing of the output stimulations as their repetitions. The notch-based modulation scheme can eliminate any complex telemetry and minimizes the power consumption. In many embodiments, as the notches only constitute a negligible portion of the Tx power, they do not degrade the efficiency of the power transfer link. In many embodiments, a VCS scheme may be adopted for better energy-efficiency, in which VDD node can be directly applied to the electrode/tissue with a controllable pulse width. In many embodiments, in replacement of a low-dropout (LDO), a simplified output voltage regulator may be used to limit the amplitude of the output stimulations within a specific range, which may further reduce the static power consumption. In many embodiments, the regulator may enable the notch-demodulation block only when the supply voltage exceeds the lower tier. On the contrary, when the supply voltage exceeds the higher tier, a discharge path may be enabled to rapidly discharge the excess incident charge. The stimulations can be delivered through a DC-block capacitor, CBCK, for charge-neutralization. In several embodiments, a discharge resistor, Rois, nulls the accumulated charge on CBCK. A light-emitting diode (LED) can be optionally included at the output. Although FIG. 8a illustrates a particular circuit architecture of an implantable treatment device, any of a variety of circuit architectures may be utilized as appropriate to the requirements of specific applications in accordance with embodiments of the invention.

In many embodiments, an implantable treatment device can be wirelessly powered and controlled by a custom Tx coil with the diameter of approximately 3 cm, as illustrated in FIG. 8b in accordance with an embodiment of the invention. In certain embodiments, a matching network ensures the impedance matching at approximately 430 MHz, the resonant frequency of the Rx energy-harvesting frontend. Although FIG. 8b illustrates a particular schematic of a Tx coil, any of a variety of architectures may be utilized as appropriate to the requirements of specific applications in accordance with embodiments of the invention.

In many embodiments, an implantable treatment device can improve the spatiotemporal resolution of the recorded signals to provide more insight into the complex mechanism of human functions. A higher spatiotemporal resolution necessitates signal recording from a smaller area with a higher recording rate. To enable recording at a fine scale, many embodiments provide implantable system-on-chips (SoCs) to realize a distributed bioelectrical recording system. The system-level requirements of such SoCs in accordance with many embodiments are long-term wireless operation, mm-sized form factor, and integration capability on a commercial CMOS process to make them scalable and cost-efficient. Miniaturizing the size of an implantable treatment device can address the needs of treatment systems since it can result in a higher sensor density and also enables signal recording at an ultra-small structural scale.

FIG. 9 illustrates a conceptual treatment system with a multi-site and distributed neural interface enabled by multiple mm-sized implantable treatment devices in accordance with an embodiment of the invention. Each implantable treatment device can include a data transceiver (TRX) that is placed on top of a microelectrode array. Because of the compact size, individual implantable treatment devices can be tightly placed on a body part (e.g., the brain) to improve the recording resolution. There are various challenges toward realizing a practical neural interface system. Accordingly, many embodiments of the treatment system address the data communication problem and provide a TRX compatible with the system-level requirements of implantable treatment systems. In many embodiments, each TRX can acts as a communication hub between the electrodes within an implantable treatment device and an external controller. Recent neural recording systems have reported up to 4096 recording electrodes. On the other hand, various recording methods such as ECOG or spike recording demand different sampling rates that may reach as high as 10 ksps per channel. Hence, the overall communication bandwidth may exceed tens of Mbps.

A practical TRX in accordance with many embodiments of the invention should support the demanded bandwidth and be compatible with most all of the system-level requirements of miniaturized implants. Achieving such a high data rate can be difficult due to severe power budget constraints and poor performance of electrically small antennas used for power-harvesting and data communication. Considering the specific absorption-rate (SAR) limit of various biological tissues and nonidealities of a WPT system such as coil misalignment and link variations, the maximum harvested power by mm-sized power harvesters can be about few hundreds of micro-Watts. Moreover, the wavelength of EM waves at the frequencies that data communication is typically conducted ranges from tens to hundreds of centimeters. A mm-sized antenna is often much smaller than the wave-length and has poor radiation efficiency. Accordingly, many embodiments provide for a TRX for mm-sized implantable treatment devices that can achieve a very high-energy efficiency to support high data rates.

Backscattering is a widely adopted technique for telemetry in implantable applications since it results in extremely low-power consumption. The transmitted data pattern can be used for load shift keying (LSK) modulation of the power coil and alters the reflected signal to an external reader. Despite achieving a superior energy efficiency over active communication, backscattering radios fail to address the main requirements of mm-sized implants. Due to the small size of the power coil and a strong power carrier, which acts as a blocker, detection of the reflected signal on the reader side may be difficult or even impossible. In addition, modulating the power coil disrupts the power flow into the system and degrade power-transfer efficiency. Furthermore, the communication bandwidth of backscattering radios is often very low due to the high-quality factor (Q) of the power coil that limits the data rate, consequently.

Active TRXs may not face the challenges of their backscattering counterparts and can potentially achieve high data rates at the expense of higher power consumptions. Considering the stringent power budget in implantable applications, a design goal of many embodiments of the treatment system can be achieving the highest possible energy efficiency; hence, proper modulation schemes can be chosen. There is a trade-off between energy efficiency and spectral efficiency in communication systems. Narrowband modulation schemes demand a relatively complex architecture to generate an accurate frequency whereas wideband modulation schemes such as on-off keying (OOK) have often less complexity and result in higher energy efficiency.

Accordingly, many embodiments provide for the design, implementation, and verification of a fully integrated and RF-powered wireless data TRX. The radio in accordance with many embodiments of the system can achieve state-of-the-art energy efficiency and the smallest form factor compared with prior mm-sized wirelessly powered active radios.

In many embodiments, the implantable treatment device can be implemented on a single CMOS silicon chip and all needed components for power delivery, energy storage, bioelectrical sensing, stimulation, and data communication, including an on-chip coil (OCC) and a dipole antenna, can be implemented on the same chip. The TRX in accordance with many embodiments of the implantable treatment device can be designed to enable simultaneous power delivery and data communication through two distinct wireless links separated in the frequency domain. In many embodiments, the implantable treatment device can support data rates of up to 2.5 Mbps in the RX and data rates of up to 150 Mbps in the TX chain, respectively. In many embodiments, the implantable treatment device can provide for fully on-chip integration that can result in cost reduction, elimination of any post-fabrication process, and reliability improvement among other benefits. Although FIG. 9 a particular system architecture of a closed-loop treatment system with several implantable treatment device for neural recording and stimulation system for neural prostheses, any of a variety of system architectures can be utilized as appropriate to the requirements of specific applications in accordance with embodiments of the invention.

While the above description contains many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. An implantable treatment device, comprising:
an energy harvesting circuit configured to harvest ambient energy, wherein the ambient energy is at least one energy selected from the group consisting of ambient electrical, magnetic energy, and electromagnetic energy;
a sensing circuit that senses bioelectrical signals and generates bioelectrical signal data;
a stimulator circuit coupled to a set of electrodes to deliver a stimulation via at least one electrode from the set of electrodes based on a set of control signals received from an external controller that determine a set of parameters of the stimulation; and
a communication circuit configured to (1) transmit to the external controller the bioelectrical signal data and (2) control the stimulator circuit to deliver energy via at least one electrode from the set of electrodes in response to the control signals received from the external controller.

2. The implantable treatment device of claim 1, wherein the bioelectrical signal is at least one signal selected from the group consisting of a neural signal, a neural LFP (local field potential), an electrocardiogram (ECG) signal, a compound action potential, an electromyogram (EMG) signal, an Electroencephalogram (EEG) signal, an Electromyogram (EMG) signal, an Electrooculogram (EOG) signal, an Electroretinogram (ERG) signal, and an Electrogastrogram (EGG) signal.

3. The implantable treatment device of claim 1, wherein the sensing circuit digitizes the bioelectrical signals to generate bioelectrical signal data.

4. The implantable treatment device of claim 1, wherein the bioelectrical signal data are used to optimize a plurality of parameters of the stimulation delivered by the stimulator circuit.

5. The implantable treatment device of claim 4, wherein the plurality of parameters includes at least one parameter selected from the group consisting of the stimulation pulse duration, amplitude of the stimulation, frequency of the stimulation, and directionality and phase response of the stimulation (mono phasic vs biphasic).

6. The implantable treatment device of claim 1, wherein the external controller processes the bioelectrical signal data to generate the set of control signals.

7. The implantable treatment device of claim 1, wherein the implantable treatment device is a closed loop neural stimulation system.

8. The implantable treatment device of claim 1, wherein the energy harvesting circuit is wirelessly powered.

9. The implantable treatment device of claim 1, wherein the bioelectrical signals are neural signal that range from 1 uV to 100 mV

10. The implantable treatment device of claim 1, further comprising filter circuitry that filters the bioelectrical signals and digitizes the filtered bioelectrical signals.

11. The implantable treatment device of claim 10, wherein the filter circuitry is designed to pass signals from 0.1 Hz to 10 KHz.

12. The implantable treatment device of claim 10, wherein the filter circuitry passes more of a frequency content of the bioelectrical signal while removing out of band noise.

13. The implantable treatment device of claim 1, wherein the bioelectrical signals are sensed within a preprogrammed time after a stimulation occurs.

14. The implantable treatment device of claim 13, wherein the preprogrammed time sensing starts at a particular time after a stimulation occurs, where the particular time is a time selected from the group consisting of 1 usec, 10 usec, 100 usec, 1 msec, 10 msec, and 100 msec after the simulation occurs.

15. The implantable treatment device of claim 1, wherein the sensing is done in an intermittent fashion with a duty cycle of less than 20%.

16. The implantable treatment device of claim 1, wherein the stimulator circuit stimulates a body part in at least one body region selected from the group consisting of peripheral nerves, spinal cord, vagus nerve, central nervous system, sacral nerve, occipital nerve, hypoglossal nerve, kidney, bladder, brain, lung, heart, muscle, and fat.

17. The implantable treatment device of claim 1, wherein the set of control signals provide a plurality of stimulation parameters for the stimulation that are optimized using processing performed in the cloud.

18. The implantable treatment device of claim 1, wherein the control signals provide a plurality of stimulation parameters that are optimized for the stimulation using data from a plurality of different patients.

19. The implantable treatment device of claim 1, wherein the control signals provide a plurality of stimulation parameters that are optimized for the stimulation with respect to a specific patient.

20. The implantable treatment device of claim 1, wherein the energy harvesting circuit comprises using energy stored within a rechargeable battery.

21. The implantable treatment device of claim 1, wherein the energy harvesting circuit comprises using energy from at least one capacitor.

22. A treatment system, comprising:
an external controller; and
a plurality of implantable treatment devices, each implantable treatment device configured for placement in, on, or adjacent an implant site, and comprising:
a plurality of electrodes;
a power harvesting circuit configured to receive energy from an external power source;
a sensing circuit coupled to a sensing/recording set of the plurality of electrodes to sense bioelectrical signals and generate bioelectrical signal data;
a stimulator circuit coupled to a stimulation set of the plurality of electrodes to deliver energy to the implant site; and
a communication circuit coupled to the sensing circuit and the stimulator circuit and comprising a transmitter circuit configured to transmit to the external controller the sensed bioelectrical signal data, and a receiving circuit configured to receive stimulation control signals from the external controller and to control the stimulator circuit to deliver energy via the stimulation set of the plurality of electrodes according to stimulation control signals.

23. The treatment system of claim 22, wherein each implantable treatment device is configured for placement in, on, or adjacent an implant site corresponding to one of: a peripheral nerve, a spinal cord, a vagus nerve, a central nervous system, a sacral nerve, an occipital nerve, a hypoglossal nerve, a kidney, a bladder, a brain, a lung, a heart, a muscle, and fat.

24. The treatment system of claim 22, wherein the external controller comprises:
a bioelectrical data processing engine configured to process bioelectrical signal data received from at least one implantable treatment device; and
a stimulation control data generator configured to generate stimulation control signals for at least one implantable treatment device based on the bioelectrical signal data.

25. The treatment system of claim 24, wherein the external controller further comprises:
a data transceiver configured to transmit the stimulation control signals to at least one implantable treatment device.

26. The treatment system of claim 25, wherein the external controller further comprises:
a power delivery circuit configured to deliver power to the plurality of implantable treatment devices.

27. The treatment system of claim 22, wherein:
the stimulation set of electrodes and the sensing/recording set of electrodes are spaced apart; and
the sensing circuit is configured to:
estimate a time window where a stimulation signal delivered by the stimulation set of electrodes arrives at the sensing/recoding set of electrodes;
determine a time based on the estimated time window; and
sense and record bioelectrical signals via the sensing/recording set of electrodes according to the determined time.

28. The treatment system of claim 27, wherein the determined time comprises one of 1 usec, 10 usec, 100 usec, 1 msec, 10 msec, and 100 msec.

29. The treatment system of claim 22, wherein:
the stimulation set of electrodes and the sensing/recording set of electrodes are spaced apart; and
the sensing circuit is configured to:
estimate a speed of the transfer of an activated neural signal from the location of the stimulation set of electrodes to the location of the sensing/recoding set of electrodes;
determine a time based on the estimated speed; and
sense and record bioelectrical signals via the sensing/recording set of electrodes according to the determined time.

30. The treatment system of claim 22, wherein:
the sensing circuit is configured to measure an evoked compound action potential (ECAP) based on bioelectrical signals sensed at the sensing/recording set of electrodes;
the communication circuit is configured to transmit the measure of ECAP to the external controller; and
the external controller is configured to adjust one or more stimulation parameters based on the ECAP, to maintain an appropriate level of neural activity.

31. A method of therapy delivery by a treatment system, the method comprising:
sensing, through at least one implantable treatment device placed at an implant site, bioelectrical activity at the implant site;
transmitting, through the at least one implantable treatment device, bioelectrical data corresponding to sensed bioelectrical activity to an external controller;
receiving, through at least one implantable treatment device placed at an implant site, stimulation control data derived at least in part from the bioelectrical data by the external controller; and
delivering, through the at least one implantable treatment device, a stimulation based on the stimulation control data.

32. The method of claim 31, wherein the at least one implantable treatment device comprises a stimulation set of electrodes spaced apart from a sensing/recording set of electrodes, and the method further comprising:
estimating a time window where a stimulation signal delivered by the stimulation set of electrodes arrives at the sensing/recoding set of electrodes;
determining a time based on the estimated time window; and
sensing/recording bioelectrical signals via the sensing/recording set of electrodes according to the determined time.

33. The method of claim 31, wherein the at least one implantable treatment device comprises a stimulation set of electrodes spaced apart from a sensing/recording set of electrodes, and the method further comprising:
estimating a speed of the transfer of an activated neural signal from the location of stimulation set of electrodes to the location of the sensing/recoding set of electrodes;
determining a time based on the estimated speed; and
sensing/recording bioelectrical signals via the sensing/recording set of electrodes according to the determined time.

34. The method of claim 31, wherein the at least one implantable treatment device comprises a stimulation set of electrodes spaced apart from a sensing/recording set of electrodes, and the method further comprising:
measuring an evoked compound action potential (ECAP) based on bioelectrical signals sensed at the sensing/recording set of electrodes;
transmitting the measure of ECAP to the external controller; and
adjusting one or more stimulation parameters based on the ECAP, to maintain an appropriate level of neural activity.

35. A treatment system, comprising:
an external treatment device configured for placement on a body part adjacent a treatment site, and comprising:
a plurality of electrodes;
a stimulator circuit coupled to the electrodes; and
a receiving circuit configured to wirelessly receive stimulation control signals and to control the stimulator circuit to deliver energy via the plurality of electrodes according to stimulation control signals;
at least one implantable treatment device configured for placement in, on, or adjacent a treatment site, and comprising:
a plurality of electrodes;
a power harvesting circuit configured to receive energy from an external power source;
a sensing circuit coupled to a sensing set of the plurality of electrodes to sense bioelectrical signals and generate bioelectrical signal data;
a communication circuit coupled to the sensing circuit and comprising a transmitter circuit configured to transmit the sensed bioelectrical signal data; and
an external controller configured to receive the sensed bioelectrical signal data from the at least one implantable treatment device, and to transmit stimulation control signals to the external treatment device.

36. The treatment system of claim 35. wherein the external treatment device comprises a sleeve configured to wrap around a patient's arm.
